(19) United States
(12) Patent Application Publication
(10) Pub. No.: US 2012/0109185 A1
(43) Pub. Date: May 3, 2012

(54) PERCUTANEOUS TISSUE GRASPING APPARATUS AND METHOD

(75) Inventors: Richard P. Fleenor, Englewood, CO (US); Robert L. Bromley, El Paso, TX (US)

(73) Assignee: PARE Surgical, Inc., Englewood, CO (US)

(21) Appl. No.: 13/284,629

(22) Filed: Oct. 28, 2011

Related U.S. Application Data
(60) Provisional application No. 61/407,752, filed on Oct. 28, 2010.

(51) Int. Cl. A61B 17/28

(52) U.S. Cl. 606/205

(57) ABSTRACT

An improved percutaneous tissue grasping apparatus and method provide for reduced trauma to tissue associated with the insertion and advancement of the apparatus. In one aspect, the percutaneous tissue grasping apparatus may include a blunt nose that is flush with or extends beyond a distal end of an outer tubular member. As such, when advanced into and through tissue, tissue is prevented from advancing proximally into the outer member. As such, the trauma attendant with advancement of such apparatus into tissue of a patent may be reduced.

### RELATED APPLICATIONS

This application claims the benefit of priority to U.S. Provisional Application No. 61/407,752 filed Oct. 28, 2010, entitled PERCUTANEOUS TISSUE GRASPING APPARATUS AND METHOD the entirety of which is hereby incorporated by reference.

### FIELD OF THE INVENTION

The present invention relates to endoscopic surgical tool apparatus and methods, and in particular, to percutaneous tissue grasping apparatus and methods.

### BACKGROUND OF THE INVENTION

Minimally invasive surgery has become the standard for many types of surgeries which were previously accomplished through open surgery. Minimally invasive surgery generally involves introducing one or more surgical instruments through a surgical or natural port in the body, advancing the one or more surgical instruments adjacent to tissue of interest, conducting the surgery with the one or more surgical instruments, and withdrawing the one or more surgical instruments from the body. In endoscopic surgery (broadly defined herein to be any surgery conducted via one or more ports, including but not limited to abdominal laparoscopy, arthroscopy, spinal laparoscopy, etc.), a port for a surgical instrument is typically made using a surgical trocar assembly. The trocar assembly often includes a port and a sharp pointed element (trocar) extending through and beyond the distal end of the port. Typically, a small incision is made in the skin at a desired location in the patient. The trocar assembly, with the trocar extending out of the port is then forced through the incision, thereby widening the incision and permitting the port to extend through the incision, past any facie, and into the body (cavity). This widening of the incision by the trocar can lead to uncontrolled dilation of the incision site (e.g., through ripping or tearing of surrounding tissue). This may lead to scarring in the area of the incision site. After widening the incision, the trocar is typically withdrawn, leaving the port in place. In certain circumstances, an insufflation element may be attached to the trocar port in order to insufflate the surgical site. A surgical instrument may then be introduced through the trocar port. Additional ports are then typically made so that additional endoscopic instruments may be introduced into the body.

While minimally invasive surgery has reduced the trauma associated with various surgical procedures and has concomitantly reduced recovery time from these surgeries, there always remains a desire in the art to further reduce the trauma to the patient.

### SUMMARY OF THE INVENTION

In view of the foregoing, a primary objective is to provide an improved apparatus and method for percutaneous insertion and tissue grasping and/or retention.

Another objective of the present invention is to provide an improved percutaneous tissue grasping apparatus and method that may be employed to enhance medical personnel efficiencies attendant to an endoscopic procedure.

An additional objective of the present invention is to provide an improved percutaneous tissue grasping apparatus and method that minimizes the associated trauma of introducing and advancing an instrument into a patient. Potential reduction in trauma may be realized through smaller introduction site wounds and an improved distal end design affecting less tissue damage.

Another objective of the present invention is to provide an improved percutaneous tissue grasping apparatus and method that can be used without additional port apparatus to reduce the trauma and associated scarring at the introduction site of the apparatus, while concurrently reducing the potential for piercing sensitive tissue structures in the patient.

Yet another objective of the present invention is to provide an improved percutaneous tissue grasping apparatus and method that is user-friendly.

An additional objective of the present invention is to provide an improved percutaneous tissue grasping apparatus that is relatively simple in construction and assembly.

An inventive percutaneous tissue grasping apparatus is provided for selective percutaneous advancement and tissue grasping/retention. In one feature, the percutaneous tissue grasping apparatus may include a tubular member, e.g., a rigid tubular member, a tissue grasper located at a distal end of the tubular member, and a handle located at a proximal end of the tubular member. The tissue grasper may be advanced and retracted relative to the tubular member between a retracted position and an advanced position. When in the retracted position, the tissue grasper may define a blunt nose. The blunt nose may be flush with or extend distally beyond the distal end of the tubular member when in the retracted position. The handle may be operative to advance and retract the tissue grasper with respect to the tubular member between the advanced and retracted positions. The blunt nose may include a continuous surface. For instance, the blunt nose may include at least a portion that is a planar surface centered about a central axis of the tissue grasper when in the closed position. The blunt nose may also have at least a portion thereof with a radius of curvature. For example, an annular edge portion of the blunt nose may be rounded (e.g., by way of a grinding process).

The tissue grasper may include first and second jaw members. The jaw members may be elastically deformable from an open position to a closed position upon retraction of the tissue grasper with respect to the tubular member. Additionally, the first and second jaw members may be biased toward an open position such that upon advancement of the tissue grasper with respect to the tubular member, the jaw members are disposed in the open position. The jaw members may be separate portions of an integral member that extends through the tubular member and is disposed for relative movement therewith between the retracted position and the advanced position.

In one embodiment, the jaw members are constructed from a shape memory alloy (e.g., Nitinol). As such, the jaw members may be elastically deformable across a wide operating temperature range, including at least a range of temperatures between standard room temperature and body temperature (e.g., between about 60 F and 105 F). The jaw members may be provided so that plastic deformation is avoided in the operating temperature range.

The percutaneous tissue grasping apparatus may advantageously limit the trauma to the tissue of a patient through which the apparatus is advanced. Tissue may be less likely to penetrate into the tubular member proximal of the distal end thereof when the apparatus is advanced through a patient's tissue. In turn, tissue is less likely to be collected by the apparatus, leading to a reduced potential for trauma at the insertion site and to tissue through which the instrument is advanced. In addition, the requisite force to advance the apparatus through the tissue of a patient may be reduced. Further still, the apparatus may have a reduced entry cross section as compared to a trocar such that the trauma to the patient at the entry site is reduced. As such, once the apparatus is removed from the patient, only a small puncture mark may remain that may heal quickly and/or have little or no scaring. The blunt nose may also reduce the potential that sensitive tissue is pierced (e.g., due to advancing the apparatus beyond the tissue of interest when introducing the apparatus).

In one aspect, the handle of the percutaneous tissue grasping apparatus may include a shaft member and a grip member extending about at least a portion of the shaft member, wherein the shaft member and grip member are disposed for selective relative movement by a user along a handle axis. One of the shaft member and the grip member may include a pawl, while the other of such members may include a rack comprising teeth spaced along the handle axis so as to define a ratchet interface.

In turn, the percutaneous tissue grasping apparatus may be provided so that upon first relative movement between the grip member and shaft member by a user, a first relative movement of the tissue grasper and the outer member between the advanced and retracted positions may be realized. This may correspond with movement of opposing portions of first and second jaw members between the open position and the closed position. Correspondingly, the ratchet interface may advantageously retain the relative position of the tissue grasper and the outer member such that opposing portions of the first and second jaw members are maintained in the closed position, while maintaining a relatively constant level of tissue grasping force. In the later regard, the degree of closure may be selectively established by a user, then maintained by the ratchet interface, wherein the user may selectively affect a desired grasping force that is sufficient to stabilize the tissue free from trauma thereto. Alternatively, the ratchet interface may retain the tissue grasper with respect to the tubular member in the retracted position.

In one approach, the handle axis may be linear, wherein relative movement of the grip member and shaft member of the handle is along such handle axis, and wherein the ratchet interface is provided along the same linear handle axis. Such an arrangement yields a compact, user-friendly percutaneous tissue grasping apparatus which may be provided to facilitate single-hand operation by a user.

In another aspect, one of the pawl and the rack may be provided to be selectively moveable laterally away from the other by a user so as to permit selective second relative movement between the grip member and the shaft member. Such second relative movement may affect second relative movement between the tissue grasper and the tubular member between a retracted and advanced position.

In a related aspect, a biasing member may be provided for applying a biasing force to oppose the first relative movement between the grip member and the shaft member. In this regard, the biasing force facilitates the ratchet interface engagement of the pawl and rack. In turn, upon relative lateral movement of the pawl and the rack away from one another, the biasing force automatically affects the second relative movement between the grip member and the shaft member, and between the tissue grasper and tubular member.

An inventive method is also provided for operating a percutaneous tissue grasping apparatus having an elongate member including a tubular member, a tissue grasper located at a distal end of the elongate member, and a handle located at a proximal end of the elongate member. The tissue grasper may be moved with respect to the tubular member between a retracted and advanced position. The method includes the steps of inserting a distal end of the elongate member percutaneously into tissue of a patient when the tissue grasper is in a retracted position such that the distal end includes a blunt nose flush with or extending beyond the distal end of the elongate member. The method further includes advancing the elongate member distally such that the distal end is adjacent to a tissue of interest. The method may include manipulating the handle to affect first relative movement of the tissue grasper with respect to the outer member from the retracted position to an advanced position. In turn, a first and second jaw member may be disposed in an open position in response to the first relative movement.

In one aspect, the method of use of a percutaneous tissue gripping apparatus may involve creating an incision in the epidermis of the patient and introducing the blunt nose of the apparatus into said patent at said incision. As such, a positive introduction location may be established prior to initial insertion of the apparatus (e.g., rather than initiating entry into the tissue with the distal end of the apparatus alone). This may allow for more precise placement of the apparatus and further reduction in trauma to the skin of the patient (e.g., reducing the potential of scarring at the introduction site).

In another aspect, the method may include second manually manipulating the handle to affect second relative movement between the tissue grasper and the outer member. Additionally, the method may include positioning opposing portions of first and second jaw members toward the other to a closed position in mechanical response to the second manually manipulating step. In turn, the tissue of interest is retained between said opposing portions of said first and second jaw members when in said closed position.

An additional aspect of the method includes withdrawing said elongate member from said patient when said tissue grasper is in the retracted position.

Numerous additional features and advantages of the present invention will become apparent to those skilled in the art upon consideration of the embodiment descriptions provided hereinbelow.

### BRIEF DESCRIPTION OF THE DRAWINGS

FIG. 1 is a perspective view of one embodiment of a percutaneous tissue grasping apparatus tool illustrating a handle, elongate member, and tissue grasper thereof in disconnected relation for purposes of illustration.

FIG. 1A is an alternate perspective view of a distal end of the elongate member and tissue grasper called out at “A” in FIG. 1 with the tissue grasper shown in an open position.

FIG. 2 is an exploded view of the handle of the percutaneous tissue grasping apparatus embodiment illustrated in FIG. 1.

FIG. 3A is a side view of the percutaneous tissue grasping apparatus embodiment of FIG. 1 with the handle, elongate member and tissue grasper thereof shown in interconnected relation.

FIG. 3B is a side cross-sectional view of the percutaneous tissue grasping apparatus embodiment shown in FIG. 3A, such cross-sectional view taken along cut line AA of FIG. 3A.

FIG. 4A is a side view of the percutaneous tissue grasping apparatus embodiment shown in FIG. 3A with a grip member of the handle shown in an advanced positioned relative to a shaft member of the handle.

FIG. 4B is a side view corresponding with the percutaneous tissue grasping apparatus embodiment shown in FIG. 3A with the grip member of the handle shown in a partially retracted position relative to the shaft member thereof to affect tissue grasping.

FIG. 4C is side view corresponding with the percutaneous tissue grasping apparatus environment shown on FIG. 3A with first and second members of the grip member advanced relative to one another to affect automatic repositioning of the grip member from the partially retracted position of FIG. 4B to the advanced position of FIG. 4A.

FIG. 5A is a side view of an alternate embodiment of a percutaneous tissue grasping apparatus in a retracted position.

FIG. 5B is a side view of the percutaneous tissue grasping apparatus of FIG. 5A in an advanced position.

FIGS. 6A-6G depict a sequence of an embodiment of a method for using a percutaneous tissue grasping apparatus.

FIG. 7 depicts the tissue grasper including first and second jaw members during a phase of manufacture of the tissue grasper.

### DETAILED DESCRIPTION

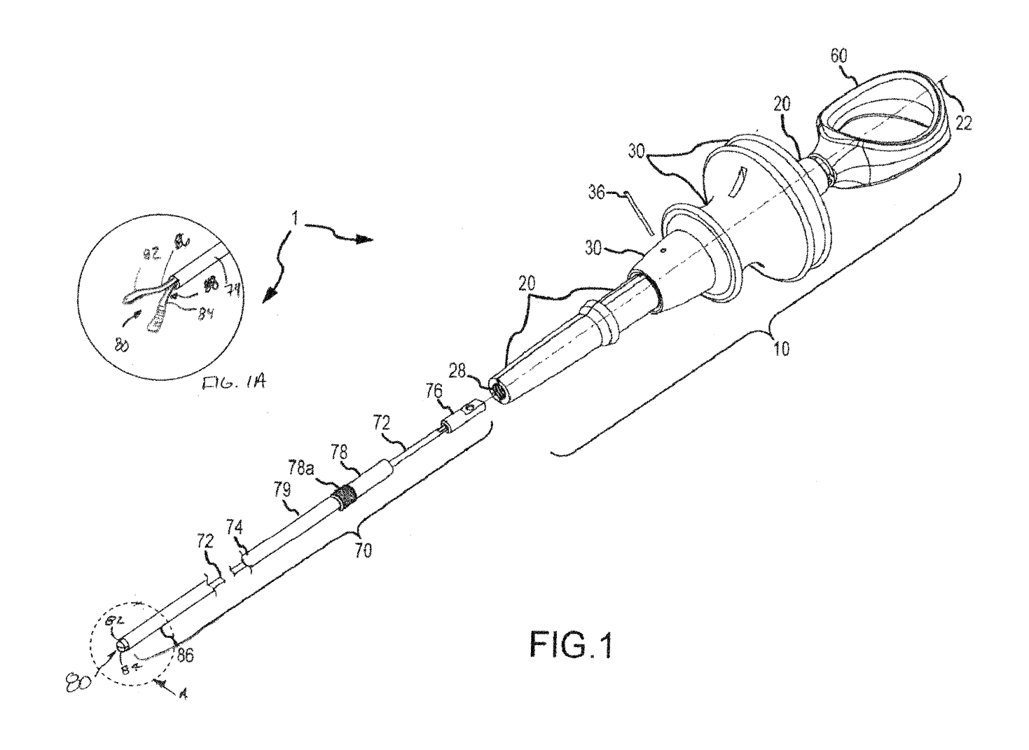

FIG. 1 illustrates one embodiment of a percutaneous tissue grasping apparatus 1 comprising a handle 10, an elongate member 70 and a tissue grasper 80. The handle 10 is interconnectable to a proximal end of the elongate member 70, and the tissue grasper 80 at a distal end of the elongate member 70.

The handle 10 may include a shaft member 20 and a grip member 30 that may extend at least partially around the shaft member 20. In the illustrated embodiment, the shaft member 20 may be provided to extend through an aperture of the grip member 30 that extends along a handle axis 22. In turn, a proximal end member 60 may be fixedly interconnected to a proximal end of the shaft member 20. As will be further described, the grip member 30 and shaft member 20 may be provided for relative movement therebetween by a user. By way of example only, the grip member 30 and shaft member 30 may be of molded plastic construction (e.g., ABS plastic, nylon plastic, etc.).

The elongate member 70 may be of a rigid construction and may include an inner member 72 (e.g., constructed of a shape memory alloy such as Nitinol) that extends through a rigid tubular outer member 74 (e.g., constructed of stainless steel), wherein the inner member 72 and outer member 74 are disposed for relative movement therebetween. In the illustrated embodiment, a proximal end of the inner member 72 may be provided with a connector 76 having an opening therethrough for interconnection to the grip member 30 via a pin 36 that extends through the grip member 30 and connector 76, wherein co-movement of inner member 72 and grip member 30 is established. The connector 76 may be integral with the inner member 72, or be attached thereto using another type of connection. Examples include but are not limited to a welded connection, a pinned connection, an interference fit, or other appropriate type of connection between the inner member 72 and the connector 76.

The tissue grasper 80 may comprise opposing first and second jaw members 82 and 84 and be moved with respect to the outer member 74 between a retracted and an advanced position. The first jaw member 82 and/or second jaw member 84 may be constructed from a shape memory alloy (e.g., Nitinol). In this regard, the first and second jaw members 82 and 84 may be separately defined portions of an integral member (e.g., the inner member 72). The first and second jaw members 82 and 84 may be provided with opposing portions disposed for selective relative movement between an open position (shown in FIG. 1A) when the tissue grasper 80 and outer member 74 are in the advanced position and a closed position (as shown in FIG. 1) when the tissue grasper 80 and the outer member 74 are in the retracted position, in which the opposing portions may be positioned in juxtaposed relation for tissue retention therebetween. The use of a shape memory alloy to construct the first and second jaw member 82 and 84 may facilitate elastic deformation of the jaw members 82 and 84 at least when in an operating temperature range. For instance, the operating temperature range may at least include the range of temperatures from room temperature (e.g., about 60 F) and human body temperature (e.g., about 105 F). Additionally, the shape memory alloy may not experience plastic deformation when in the operating temperature range. Thus, deformation of the jaw members 82 and 84 may be entirely elastic such that the original shape imparted to the jaw members 82 and 84 may be restored despite substantial deformation of the jaw members 82 and 84 (i.e., plastic deformation may be avoided).

In one embodiment, the jaw members 82 and 84 may be separate portions of an integral member. As such, the inner member 72 may integrally include the separate jaw members 82 and 84. The first and second jaw members 82 and 84 may be formed using an electrical discharge machining (EDM) technique such as a wire EDM technique. As shown in FIG. 7, a pilot hole 700 may be formed at the proximal base of the jaw members 82 and 84. This pilot hole 700 may be formed using a small hole drilling EDM process. From the pilot hole 700, a wire-cut EDM slot 702 may be formed defining the opposing faces of the first and second jaw member 82 and 84 (e.g., including teeth or other opposing surface features). The slot 702 may extend to a distal portion 704 of the slot 702 that is spaced apart from the distal end of the jaw members 82 and 84. The thickness of the slot 702 at the proximal end thereof adjacent to the pilot hole may dictate the force required to elastically deform the first and second jaw members 82 and 84 into a closed position once set into a final position as will be discussed below.

In turn, the distal tip 706 of the jaw members 82 and 84 may be removed (e.g., using a grinding technique) to free the first jaw member 82 from the second jaw member 84 at separation line 708. The first and second jaw members 82 and 84 may be formed into an outwardly biased position (e.g., as shown in FIG. 1A) by placement of the jaws into a fixture such that the jaws assume the shape shown in FIG. 1A. The jaw members 82 and 84 may then be heated above the transition temperature of the shape memory alloy used to construct the jaw member (e.g., approximately 950° F.). Once heated, the jaw members 82 and 84 may be quenched to a temperature below the transition temperature such that the first and second jaw members 82 and 84 remain in an outwardly biased position shown in FIG. 1A. As previously mentioned, the shape memory alloy used to construct the first and second jaw members may provide for elastic deformation exclusively at operating temperatures such that the jaw members are not plastically deformable at operating temperatures.

In this regard, upon first relative movement of the first and second jaw members 82 and 84 with respect to the distal end of the outer member 74 from the retraced position to the advanced position, the first jaw member 82 and/or second jaw member 84 may be elastically displaced away from the other of the first jaw member 82 and/or second jaw member 84 such that the first and second jaw members 82 and 84 are disposed in the open position.

Upon second relative movement of the first and second jaw members 82 and 84 with respect to the outer member 74 from the advanced position to the retracted position, the jaw members may be retracted into the outer member 74 into the retracted position shown in FIG. 1 wherein the first and second jaw members 82 and 84 are displaced into the closed position by way of the outer member 74 moving with respect to the jaw members 82 and 84. That is, the relative movement of the outer member 74 with respect to the jaw members 82 and 84 between advanced and retracted positions may act to compress the jaw members into the closed position.

In this regard, the first jaw member 82 and second jaw member 84 may have corresponding first cam surface 86 and second cam surface 88. The first cam surface 86 and second cam surface 88 may cooperate with the distal end of the outer member 74 such that when the tissue grasper 80 is moved in a second relative manner with respect to the outer member 74 from the advanced to retraced position, the interaction of the outer member 74 and the first and second cam surfaces 86 and 88 act to compress the jaw members 82 and 84 into a closed position shown in FIG. 1.

In one embodiment, the distal end of the outer member 74 terminates in a substantially circular opening, the diameter of which extends generally perpendicular to the relative motion of the tissue grasper 80 with respect to the outer member 74. In this regard, upon second relative movement of the tissue grasper 80 with respect to the outer member 74 from the advanced position to the retraced position a similar amount of compressive force may act upon the first jaw member 82 and the second jaw member 84, thus resulting in substantially equal amounts of deflection with respect to each jaw member 82 and 84. In this regard, when the tissue grasper 80 is moved relative to the outer member 74 from the advanced position to the retracted position, the first and second jaw members 82 and 84 may undergo a similar amount of deflection such that the jaw member substantially close such that the opposing faces thereof are aligned with a longitudinal axis of the elongate member 70 extending along the handle axis 22.

In contrast, previous tissue grasping apparatus may include a piercing tip at the distal end of the elongate member 70. In such tissue graspers, the piercing tip of the elongate member 74 may result in cam services 86 and 88 contacting the outer member 74 at different relative locations along the length of the outer member 74 when being retracted. In this regard, the first and second jaw members 82 and 84 may be displaced off axis such that the closure of the first and second jaw members 82 and 84 does not occur along an axis parallel with the handle axis 22. This may result in tissue being displaced away from the handle axis 22 being grasped by the tissue grasper 80. Such displacement of tissue is caused by unequal compressive forces applied by a piercing tip that may cause unwanted displacement of the tissue grasper 80. As such, the control of the tissue may be compromised in that the closure action may cause an off axis displacement of the tissue grasper 80 when being positioned in a closed position.

In the retracted position as shown in FIG. 1, the first and second jaw members 82 and 84 may define a blunt nose or surface that is flush with or extends beyond the distal end of the outer member 74. The first and second jaw members 82 and 84 may be sized appropriately to substantially occupy an entirety of the inner diameter (e.g., corresponding to the diameter of the substantially circular opening) of the outer member 74. In this regard, the first and second jaw members 82 and 84 may cooperate with the outer member 74 to present a substantially continuous distal end of the elongate member 70 such that substantially no openings or cavities exist at the distal end of the elongate member 70 when the first and second jaw members 82 and 84 are in the retracted position. As such, a continuous surface may be presented that extends across the whole distal end of the jaw members 82 and 84 when in the retracted position. The first and second jaw members 82 and 84 may cooperate to define the continuous surface. The continuous surface may extend along a side portion of the first and second jaw members 82 and 84 when in a closed position (e.g., proximally along a side of the closed first and second jaw members 82 and 84).

The blunt nose or surface of the first and second jaw members 82 and 84 may include at least a portion comprising a planar surface. The planar surface may be at a distal end of the first and second jaw members 82 and centered about a central axis of the first and second jaw members 82 and 84 when in a closed position. At least a portion of the blunt nose may include a radius of curvature. For example, the blunt nose may include rounded edges about an annular periphery thereof (e.g., produced by way of a grinding process). The first and second jaw members 82 and 84 may be interconnected to, or provided integrally with, the inner member 72 of the elongate member 70, wherein relative movement of the inner member 72 and outer member 74 between the advanced and retracted position affects movement of the first and second jaw members 82 and 84 as described above.

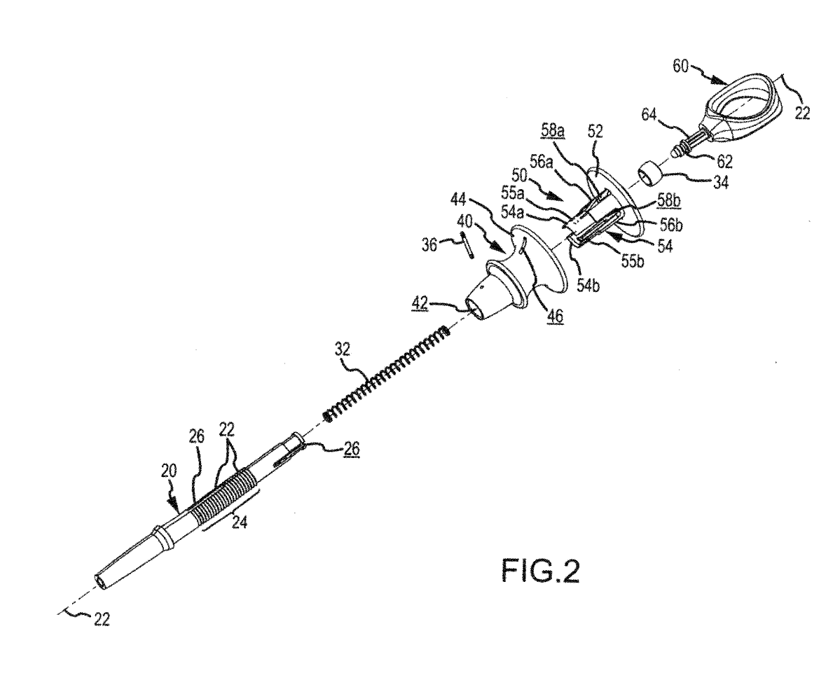

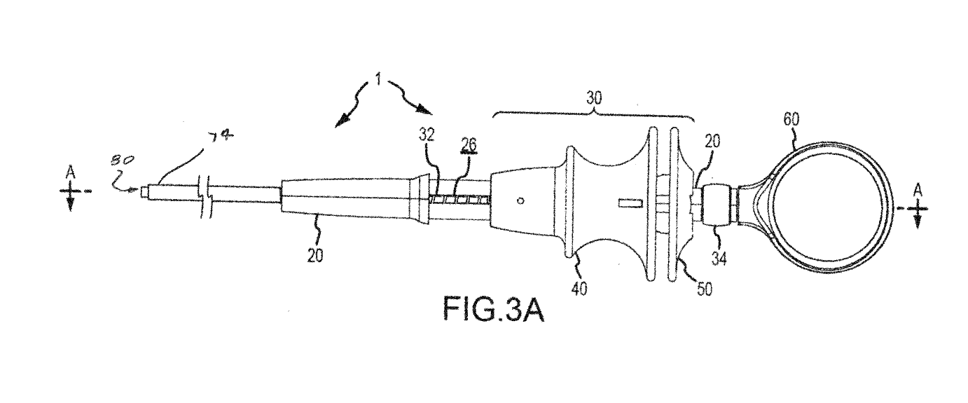

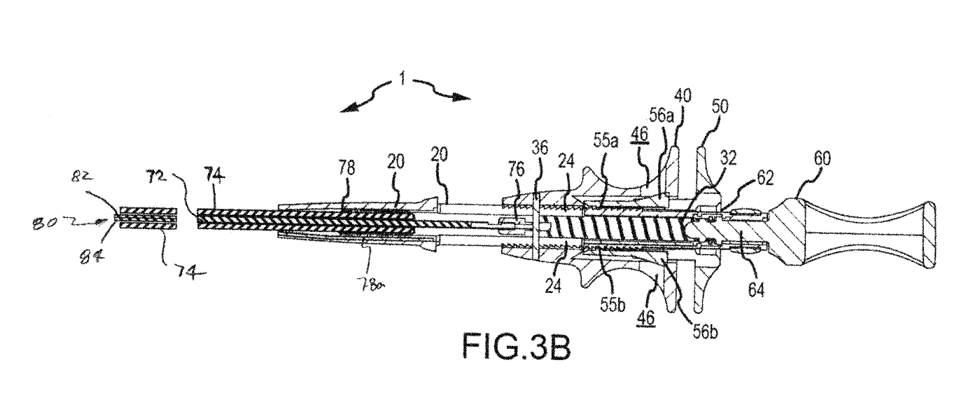

Reference is now made to FIG. 2 and FIGS. 3A and 3B. The shaft member 20 may include a plurality of teeth 22 spaced along a length of an outer surface of the shaft member 20 to define a rack 24. In one approach, rack 24 may be defined by an insert molded metal portion. In the illustrated embodiment, the teeth 22 may each extend about an annular periphery of the shaft member 20. The shaft member 20 may further include an elongate slot 26 for receipt of the above-noted pin 36 therethrough. As will be further described, the pin 36 may move within and along the elongated slot 26 during relative movement of the shaft member 20 and grip member 30.

With further reference to FIG. 2, the grip member 30 may comprise a first member 40 and a second member 50. The first member 40 may include a central aperture 42 that extends therethrough and a laterally extending flange portion 44 near a proximal end thereof. In turn, the second member 50 may include a laterally extending flange portion 52 and a longitudinally extending connector portion 54 that is interconnectable within the aperture 42 of the first member 40. The connector portion 54 may include a plurality of legs extending away from the flange portion 52 for interconnected positioning within the aperture 42. In the illustrated embodiment, two opposing leg members 54a and 54b are illustrated.

Each of the leg members 54a and 54b may be provided with a corresponding connector tab 56a and 56b, respectively, each of which are cantilevered within a corresponding window 58a and 58b, respectively. The connector tabs 56a and 56b may each comprise an outer surface that tapers outwardly away from a distal end to a proximal end thereof. In turn, upon insertion of the connector portion 54 into the aperture 42 of the first member 40, the cantilevered tabs 56a and 56b may elastically deflect inward and then back outward as tabs 56a and 56b advance into snap-fit engagement corresponding apertures 46 of the first member 40. As will be further described, each of the leg members 54a and 54b may each be further provided with a corresponding inwardly-extending, tooth-shaped pawl 55a (shown in phantom lines in FIG. 2) and 55b respectively, located in aligned distal relation with distal ends of the cantilevered tabs 56a and 56b, respectively.

With further reference to FIG. 2, the handle 10 may further comprise a biasing member 32 (e.g., a metal spring) for positioning within the shaft member 20 and grip member 30. In the illustrated embodiment upon assembly, a proximal end of the biasing member 32 may be positioned within a corresponding retention notch 62 provided on a stem 64 of the proximal end member 60. A distal end of the biasing member 32 may abut pin 36.

When assembled, the stem 64 of the proximal end member 60 may be inserted into a proximal end portion of the shaft member 20. To facilitate such insertion, the proximal end portion of the shaft member 20 may include one or more slits 26 extending distally from the proximal end to allow for elastic deformation of the proximal end portion of the shaft member during receipt of the stem 64 therewithin. After insert positioning of the stem 64 within the proximal end portion of the shaft member 20, a resilient retention band 34 may be disposed in overlapping relation to the proximal end portion of the shaft member 20 and stem 64 of the proximal end portion member 60 to maintain an axially fixed relation therebetween. In the illustrated arrangement, the proximal end member is axially fixed relative to shaft member 20 that may rotate to an extent relative thereto.

With further reference to FIGS. 1, 3A and 3B, the elongate member 70 may include an interconnect member 78 disposed about a length of the inner member 72 near the proximal end thereof. In turn, the interconnect member 78 may be located within a distal end portion of the shaft member 20 upon assembly, wherein an outer threaded surface portion 78a may rotatively engage and interconnect to an inner threaded surface portion 28 of shaft member 20.

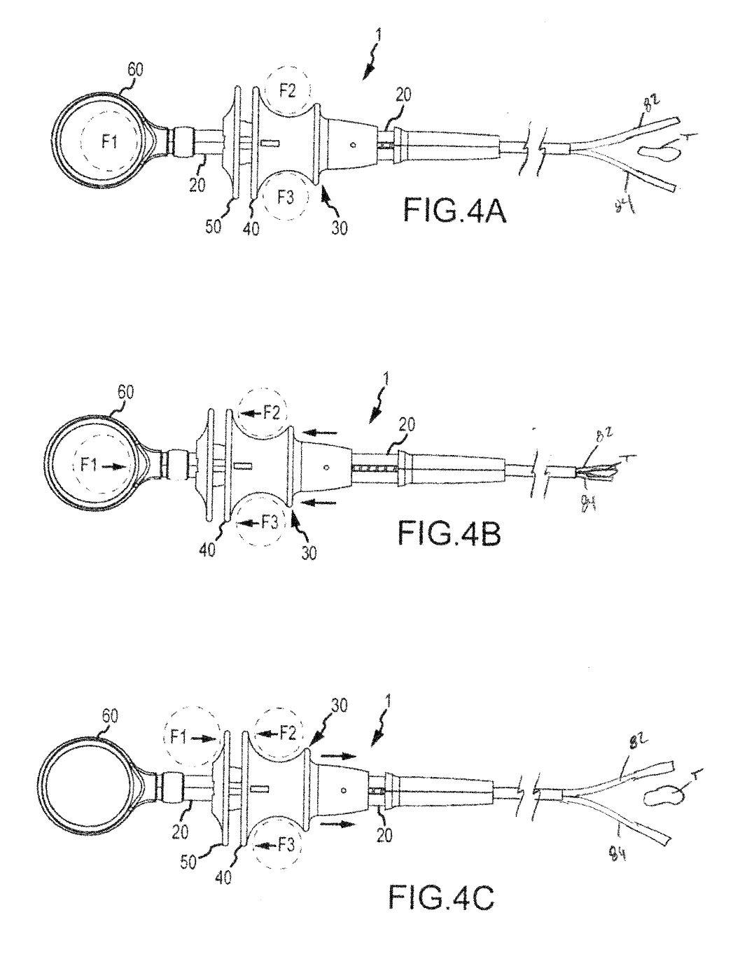

Operation of the percutaneous tissue grasping apparatus 11 will now be described with reference to FIGS. 4A-4C and FIG. 3B. The handle 10 may affect movement of the tissue grasper 80 between the retracted and advanced position. FIG. 4A shows handle 10 with grip member 30 in a first advanced position relative to the shaft member 20. In the advanced position the first and second jaw members 82 and 84 are in an advanced position and open with respect to each other for locating tissue T therebetween. For such tissue locating, the tool 1 may be manipulated via fingers F1, F2 and F3 of a user positioned as shown.

FIG. 4B illustrates retraction of the grip member 30 relative to the shaft 20 to a partially retracted position so as to close the first and second jaw members 82 and 84 to a closed position with tissue T grasped therebetween. For such retraction, the tool may be manipulated via positioning of fingers F1, F2 and F3 as shown, wherein finger F1 may be advanced relative to fingers F2 and F3 and/or wherein fingers F2 and F3 may be retracted relative to finger F1 (e.g., a squeezing or pinching action). The same action could be continued such that the tissue grasper 80 is moved with respect to the outer member 74 to the retracted position as shown in FIG. 1. In relation to a partially retracted position of the grip member 30 relative to the shaft member 20, the pawls 55a and 55b of the second member 50 of the grip member 30 may interface with the rack 24 of the shaft member 20 in a ratchet-like manner, wherein the grip member 30 may be retained in the second position shown in FIG. 4B. In this regard, the partially retracted positioning of grip member 30 is illustrated in FIG. 3B. As shown, the pawls 55a and 55b are disposed in opposing engaged relation with the teeth 22 of the rack 24. By virtue of the positioning of the grip member 30, biasing member 32 may be compressed proximal to pin 36, wherein a distally-directed biasing force may applied by the biasing member 32 to the grip member 30.

To release the grip member 30 from the retracted position, the flange portion 52 of the second member 50 may be distally advanced relative to the flange portion 42 of the first member 40. In turn, upon such advancement a tapered leading edge surface at the distal end of each of the leg members 54a and 54b may interface with opposingly-tapered, leading edge surfaces provided about an inward ledge 48 within the aperture 42 of the first member 40. Upon advancement of the flange portion 52 of the second member 50 relative to the flange portion 42 of the first member 40, the tapered leading edge surface at the distal end of each of the leg member 54a and 54b may advance distally with respect to the inward ledge having opposing-tapered surfaces. Accordingly, the relative distal movement of the leading edge surfaces of the leg members 54a and 54b may result in a lateral movement of the leg members 54a and 54b with respect to the shaft member. That is, the interface of the noted tapered surfaces forces each of the leg members 54a and 54b to elastically deflect outward to a sufficient degree to permit pawls 55a and 55b to disengage from the teeth 22 of the rack 24, wherein the biasing force of biasing member 32 automatically returns the grip member 30 from the partially retracted position shown in FIG. 4B back to the advanced position shown in FIG. 4C to effect movement of the tissue grasper 80 with respect to the outer member 74 to an advanced position.

As used herein, a retracted position refers to a position of a tissue grasper and the outer member corresponding to a first extent of relative travel between the grip member and the shaft member. In this regard, the first and second jaw members 82 and 84 may be in a closed position when in the retracted position (e.g., as shown in FIG. 1). Additionally, the jaw members 82 and 84 may also be positioned into a closed position when the handle 10 is in a partially retracted position (e.g., wherein the grip and shaft members are not at a first extent of relative travel). In turn, the first and second jaw members 82 and 84 may be in the closed position when the handle 10 is in a partially advanced position. When the handle 10 effects relative movement of the tissue grasper and outer member to the retracted position, the first and second jaw member 82 and 84 may be positioned such that a blunt nose is defined as discussed above. As such, the fully retracted position may correspond to the first and second jaw member 82 and 84 being in a closed position to define a blunt nose flush with or extending distally of the outer tubular member 74.

To realize tissue release by first and second jaw members 52a and 52b, fingers F1, F2 and F3 of a user may be positioned as shown in FIG. 4C. Then, finger F1 may be advanced relative to fingers F2 and F3 and/or fingers F2 and F3 may be retracted relative to finger F1 so as to release the ratchet interface (e.g., a squeezing or pinching action), wherein the biasing member 32 functions to automatically advance the grip member 30 so as to open the first and second jaw members 82 and 84.

In relation to the described percutaneous tissue grasping apparatus embodiment 1, the tissue grasper 80 may comprise known arrangements in which opposing jaw members are displaced between open/closed positions in response to the advancement/retraction or retraction/advancement of an interconnection member that extends to a handle operable by a user.

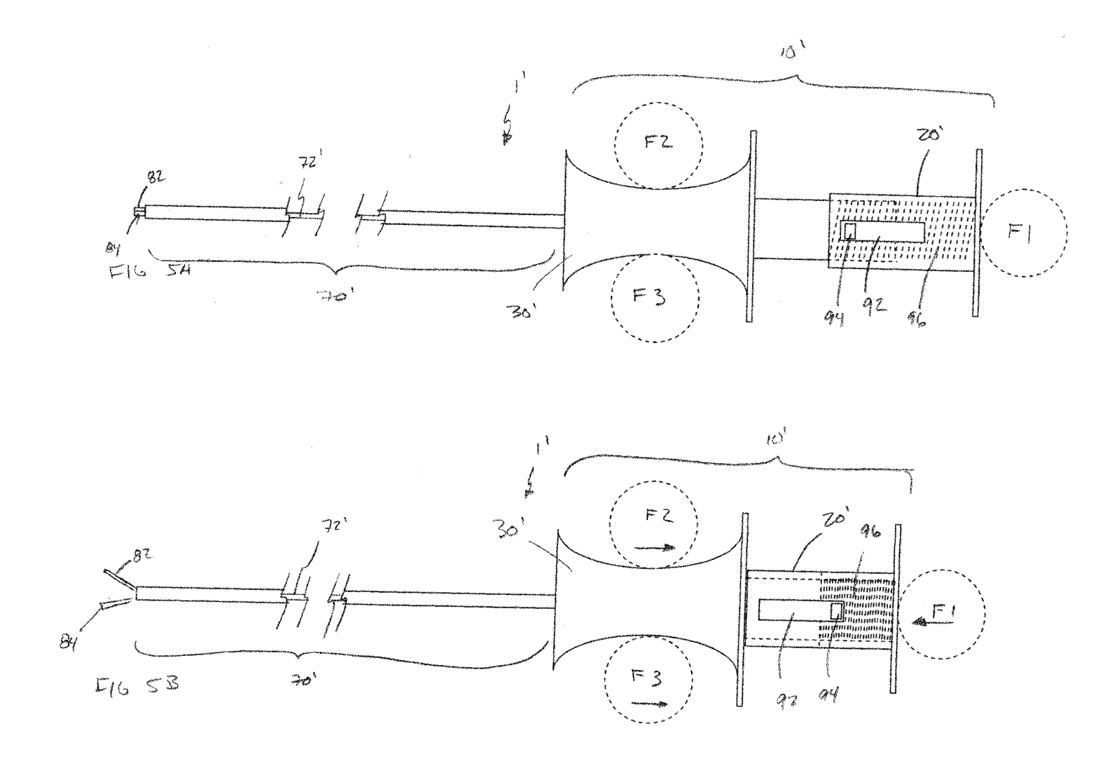

With reference to FIG. 5A, an alternate embodiment of a percutaneous tissue grasping apparatus 1′ is shown. The tissue grasping apparatus 1′ may include an outer member 74′ with a corresponding inner member 72′ extending therethrough in a similar manner as described above with regards to FIG. 1, FIG. 2, FIG. 3A, and FIG. 3B. The first jaw member 82 and the second jaw member 84 are shown in a closed position in FIG. 5A. The tissue grasping apparatus 1′ may include a handle 10′. The handle 10′ may include a grip member 30′ and a shaft member 20′. The grip member 30′ may be operatively interconnected to the outer member 74′. The shaft member 20′ may be operatively interconnected to the inner member 72′, which in turn is connected to the first and second jaw members 82 and 84. A biasing member 96 may be provided in order to bias the shaft member 20′ with respect to the grip member 30′ to a retracted position shown in FIG. 5A. A slot 92 may be defined in the shaft member 20′ that cooperates with a tab 94 extending from the grip member 30′. As such, the cooperation of the slot 92 and tab 94 may limit the travel of the shaft member 20′ proximal with respect to the grip member 30′ under the force of the biasing member 96 to the retracted position.

With additional reference to FIG. 5B, a user may position fingers F2 and F3 as shown with respect to the grip member 30′ in FIG. 5B. The user may position a finger F1 proximal to the shaft member 20′. In this regard, the user may impart relative movement between fingers F1 and fingers F2 and F3 in order to overcome the bias force imparted by the bias member 96 in order to dispose the grip member 30′ and shaft member 20′ in an advanced relative position shown in FIG. 5B. As such, the relative movement of the grip member 30′ with respect to shaft member 20′ may also result in relative movement of the inner member 72′ with the outer member 74′ such that the first jaw member 82 and second jaw member 84 are disposed in an open position as shown in FIG. 5B. Upon release of the force imparted by the user, the bias member 96 may act upon the shaft member 20′ and grip member 30′ so as to return the apparatus 1′ to the arrangement shown in FIG. 5A wherein the bias member 96 biases the grip member 30′ and shaft member 20′ in a retracted position as shown in FIG. 5A. Of note, in FIG. 5A, the first jaw member 82 and the second jaw member 84 define a blunt nose extending proximally beyond the outer member 74′ when the apparatus is in the retracted position shown in FIG. 5A.

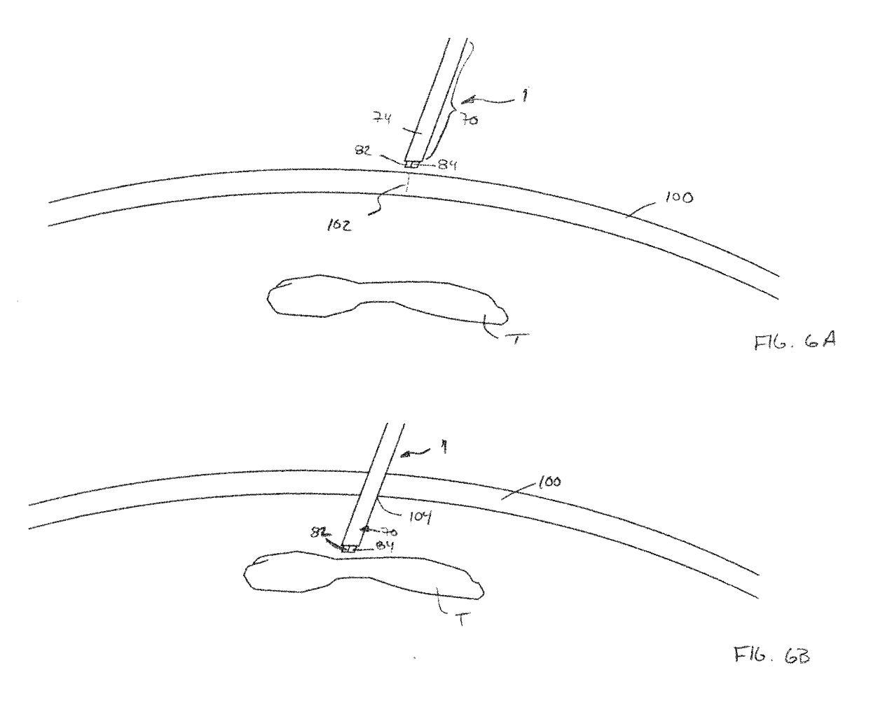

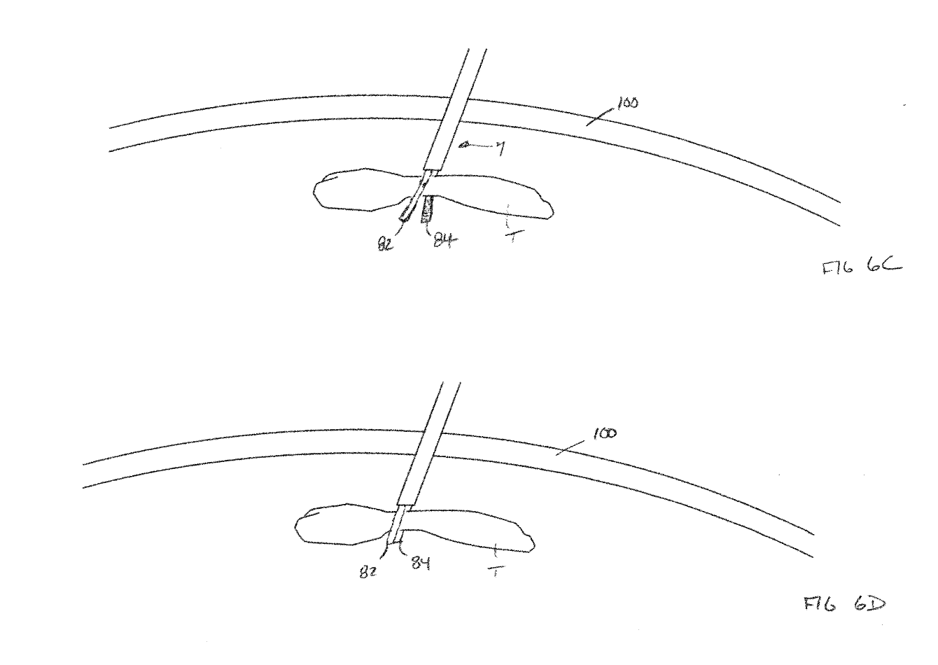

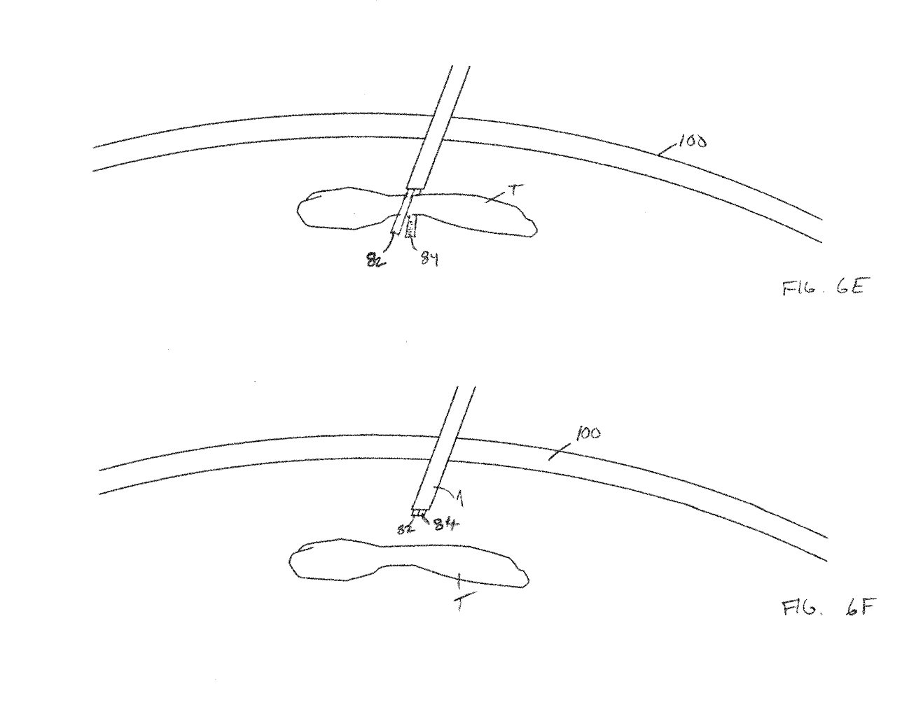

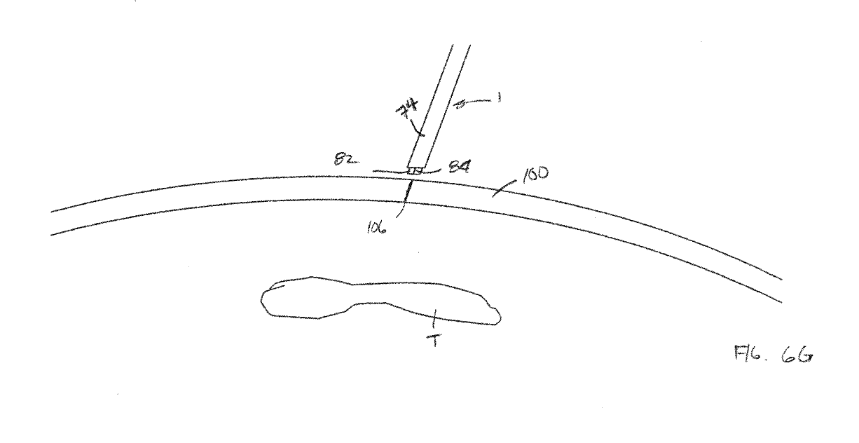

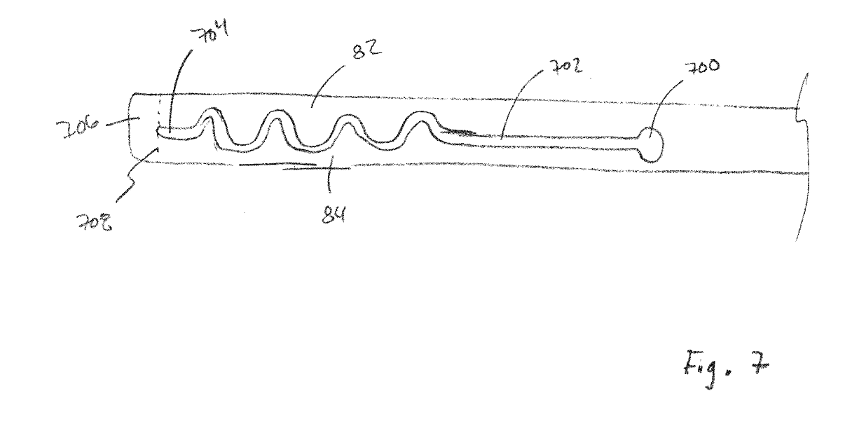

Turning to FIG. 6A-6G, a progression depicting one method of use of the percutaneous tissue grasping apparatus is depicted. In FIG. 6A, a tissue grasping apparatus 1 is disposed adjacent to the skin 100 of the patient such that the tissue grasper 80 is disposed relative to the outer member 74 in a retracted position. As shown, the first jaw member 82 and second jaw member 84 define a blunt nose extending beyond the outer member 74. Alternatively, the first jaw member 82 and second jaw member 84 may also be provided flush with the distal end of the outer member 74. Tissue of interest T may be disposed below the skin 100 of the patient. An optional incision 102 may be made in the skin 100 to prepare an introduction site for the percutaneous tissue grasping apparatus 1.

In any regard, the percutaneous tissue grasping apparatus is advanced percutaneously through the skin 100 such that the distal end of the elongate member 70 is disposed adjacent to the tissue of interest T. It should be noted that upon advancement of the percutaneous tissue grasping apparatus 1 through the skin 100 and other tissue of the patient, the blunt nose of the apparatus 1 comprised of the first and second jaw members 82 and 84 reduces the potential that any tissue is advanced proximally into the outer member 74. This may result in decreased effort to advance the apparatus 1 through the tissue of the patient and less trauma to the area. Additionally, the blunt nose may reduce the potential for inadvertent piercing of other tissue upon advancement.

Once the percutaneous tissue grasping apparatus 1 is advanced such that the distal end of the elongate member 70 is adjacent to the tissue of interest T, the first and second jaw members 82 and 84 may be placed in an open position as depicted in FIG. 6C by moving the tissue grasper 80 with respect to the outer member 74 to an advanced position. With reference to the foregoing, the first and second jaw members 82 and 84 may be advanced by relative movement of the grip member and a shaft member of a handle portion (not shown).

The tissue grasping apparatus 1 may be positioned such that the first and second jaw members 82 and 84 are disposed with the tissue of interest T disposed therebetween. Subsequently, the first and second jaw members 82 and 84 may be positioned in a closed position as shown in FIG. 6D such that the tissue of interest T is disposed therebetween. A number of various instruments (not shown) may also be inserted through the skin 100 in order to perform a desired operation on the tissue of interest T. Once all desired operations have been completed, the first and second jaw members 82 and 84 may be again displaced in an open position as shown in FIG. 6E by moving the tissue grasper 80 with respect to the outer member 74 to an advanced position. The percutaneous tissue grasping apparatus 1 may be withdrawn such that the tissue T is no longer disposed between the first and second jaw members 82 and 84 such that they can be positioned in a closed position when the tissue is no longer between the two jaw members 82 and 84 as shown in FIG. 6F by moving the tissue grasper 80 with respect to the outer member 74 to a retracted position. In this regard, the percutaneous tissue grasping apparatus 1 may then be withdrawn from the skin 100 of the patient.

Of note, no additional devices need to be introduced through the skin 100 of a patient (e.g., trocars, ports, etc.) or used when inserting the percutaneous tissue grasping apparatus 1 through the skin 100 of the patient. In turn, the resulting wound site 106 may be much smaller (e.g., the wound site 106 may be a small puncture wound) than that associated with a trocar or other port assembly asserted into the skin 100 to facilitate advancement of an instrument therethrough. Additionally, in that the percutaneous tissue grasping apparatus 1 includes a blunt nose provided flush with or advanced beyond the outer member 74 of the elongate member 70, the amount of tissue damage at the wound site 106 may be reduced in that tissue may not be introduced into the outer member 74 proximal of the distal end thereof. As such, the patient may undergo less trauma such that there is less discomfort and pain associated with such a procedure and a speedier recovery of the wound site. Further still, there may be little or no scarring at the wound site 106 when using the apparatus described herein.

In another arrangement, it has been recognized that an apparatus may include jaw members with a sharpened distal end. In this regard, this arrangement may benefit from the advantages associated with jaw members constructed of a shape alloy material. The sharpened distal end of the jaw members may extend beyond a distal end of an outer member when in the retracted position. Alternatively, the sharpened distal portion may be fully nested within the outer member when in a retracted position.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain known modes of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

1. A percutaneous tissue grasping apparatus, comprising:
an elongate member including a rigid tubular outer member;
a tissue grasper located at a distal end of said elongate member, wherein the tissue grasper is disposed for relative movement with respect to the rigid tubular outer member between an advanced and retracted position;
a handle located at a proximal end of said elongate member and including a shaft member and a grip member, said shaft member and said grip member being disposed for first relative movement by a user along a handle axis to permit first relative movement between said tissue grasper and said rigid tubular outer member from said advanced position to said retracted position;
wherein when in said retracted position, said tissue grasper defines a blunt nose flush with or extending beyond a distal end of said outer member.

2. The apparatus according to claim 1, wherein the blunt nose includes a continuous surface.

3. The apparatus according to claim 2, wherein at least a portion of the continuous surface includes a planar surface centered about a central axis of the tissue grasper when in the closed position.

4. The apparatus according to claim 3, wherein at least a portion of the continuous surface includes a radius of curvature.

5. The apparatus according to claim 4, wherein an annular edge portion of the tissue grasper includes said radius of curvature.

6. The apparatus according to claim 5, wherein said radius of curvature is defined by grinding said tissue grasper.

7. The apparatus according to claim 6, wherein said tissue grasper includes a first jaw member and a second jaw member, wherein opposing portions of said first and second jaw members are disposed for relative movement between an open and closed position, wherein tissue is graspable between said first and second jaw members in said closed position.

8. The apparatus according to claim 7, wherein said first and second jaw members are elastically deformable from said open position and said closed position upon retraction of the tissue grasper with respect to the rigid tubular outer member to the retracted position.

9. The apparatus according to claim 8, wherein the first and second jaw members are biased toward said open position upon advancement of said tissue grasper with respect to said rigid tubular outer member to the advanced position.

10. The apparatus according to claim 9, wherein the first and second jaw members comprise separate portions of an integral member extending through the rigid tubular outer member.

11. The apparatus according to claim 10, wherein the first and second jaw members are constructed from a shape memory alloy.

12. The apparatus according to claim 11, wherein the shape memory alloy is a nickel titanium alloy.

13. The apparatus according to claim 12, wherein the first and second jaw members are elastically deformable and not plastically deformable between an operating temperature range.

14. The apparatus according to claim 13, wherein the operating temperature range includes at least standard room temperature and human body temperature.

15. The apparatus according to claim 14, wherein said axis handle is linear.

16. An apparatus as recited in claim 15, said handle further comprising:
a biasing member for applying a biasing force to oppose said first relative movement between said grip member and said shaft member, wherein upon said selective lateral movement said biasing force automatically affects a second relative movement between said tissue grasper and said rigid tubular outer member.

17. The apparatus according to claim 16, wherein said grip member extends about at least a portion of said shaft member, and wherein one of said grip member and said shaft member includes a pawl and the other of said grip member and said shaft member includes a rack comprising a plurality of teeth spaced along said handle axis to define a ratchet interface therebetween, and wherein said ratchet interface maintains said handle in one of said retracted and advanced positions.

18. An apparatus as recited in claim 17, wherein said selective lateral movement results from a tapered leading edge surface of a first portion of said grip member advances relative to an opposingly-tapered surface of a second portion of said grip member.

19. An apparatus as recited in claim 18, wherein said first relative movement and said second relative movement between said grip member and said handle member is completable by a user via one-hand operation of the apparatus.

20. An apparatus as recited in claim 19, said grip member including first and second members interconnectable and disposed for selective relative movement therebetween, wherein first relative movement between said first and second members by a user affects said lateral movement of one of said pawl and said rack away from the other one.

21. An apparatus as recited in claim 20, wherein said first and second members include corresponding first and second lateral flange portions extending away from said handle axis in opposing relation to each other.

22. An apparatus as recited in claim 21, said handle further comprising:
a proximal end member fixedly interconnected to a proximal end of said shaft member, wherein said proximal end member is advanceable by a user to affect said first relative movement between said grip member and said shaft member.

23. An apparatus as recited in claim 22, wherein said first lateral flange portion is engageable by one or more digits of a user's hand and said proximal end member is engageable by another digit of said user's hand to affect said first relative movement.

24. An apparatus as recited in claim 23, wherein said first lateral flange portion is engageable by one or more digits of a user's hand and said second lateral portion is engageable by another digit of said user's hand to affect said second relative movement.

25.-30. (canceled)
